(19) United States
(12) Patent Application Publication
(10) Pub. No.: US 2010/0106075 A1
(43) Pub. Date: Apr. 29, 2010

(54) Method for iontophoretic fluid delivery

(75) Inventor: Ashok V. Joshi, Salt Lake City, UT (US)

Correspondence Address: Kent S. Burningham, Esq.;KENT S. BURNINGHAM, P.C., 466 South 500 East, Salt Lake City, UT, 84102, US

(73) Assignee: Jamal S. Yanaki

(21) Appl. No.: 12/587,566

(22) Filed: Oct. 7, 2009

Related U.S. Application Data
(63) Continuation of application No. 11/626,254, filed on Jan. 23, 2007, now abandoned.

(51) Int. Cl. A61N 1/30

(52) U.S. Cl. 604/20; 604/501

(57) ABSTRACT

A method is provided for low cost, accurate, iontophoretic fluid delivery. The method includes providing an electronic circuit coupling a plurality of electrodes, charging a chargeable electromotive cell to a selected potential and/or charge in response to a selected quantity of beneficial agent to be delivered, the chargeable electromotive cell being electronically coupled with the electronic circuit, applying the selected quantity of beneficial agent to at least one electrode, placing the at least one electrode in contact with skin, and delivering the selected quantity of beneficial agent. The method may also include preparing the skin using a skin preparation device in order to enhance the delivery of the beneficial agent.

### RELATED APPLICATIONS

This is a continuation patent application of pending prior U.S. patent application Ser. No. 11/626,254 of Ashok V. Joshi for METHOD FOR IONTOPHORETIC FLUID DELIVERY that was filed on Jan. 23, 2007.

### FIELD OF THE INVENTION

The present invention relates to an apparatus and methods for delivering drugs or other beneficial agents. More specifically, the present invention relates to iontophoretic electrotransport devices and methods of their use in delivering treatment to a body.

### BACKGROUND OF THE INVENTION

Iontophoretic transport of drug or biological treatments is well known, and is commonly used as one way to transport such treatments across a surface and into a body. Many iontophoretic devices have been developed, as witnessed by the quantity of issued patents and pending applications mentioning such phenomena.

Existing iontophoretic devices may generally be classified into two groups based upon their electromotive source. The first such group may be characterized as disposable, and are driven by a galvanic or electrochemical reaction encompassing electrodes bathed in an electrolyte carrying the treatment ions and offering a relatively low voltage. Such devices inherently require long treatment time intervals and are also generally constructed to be inexpensive, used once, and then thrown away. The second type of iontophoretic device typically is driven by an auxiliary power module. While treatment time requirements for devices having auxiliary power modules are generally reduced, the power modules are expensive, and so typically must be reused.

FIG. 1 is a schematic block diagram illustrating one embodiment of a disposable device 100 in accordance with the prior art. The disposable device 100 may be constructed on an adhesive strip 102. Cationic chamber 104 and anionic chamber 106 are formed in the adhesive strip 102 to create separated volumes in which to house cationic and anionic treatment materials, respectively. An electrolytic cell created by a chemical reaction between the cationic and anionic electrodes in an electrolyte provides the electromotive force to operate the device for ion transfer to a patient. A first electrode 108 installed in the cationic chamber and a second electrode 110 installed in the anionic chamber are connected by a conductor 112 to form an electron transporting leg of an electric circuit. Application of the adhesive strip to a human body completes the circuit, and initiates a flow of treatment ions through the patient's skin.

An electrode 108 may be formed from zinc, with an electrode 110 being made from silver chloride. The electrolyte contained in the cationic chamber 104 and anionic chamber 106 directly contacts the skin to be treated, and necessarily is limited in reactivity to avoid skin irritation. Conductive salt solutions (such as 1% NaCl) commonly are employed as electrolytes due to their compatibility with a patient's skin. A device 100, as described, will generate an electromotive force for ion transfer totaling about 1 Volt. In use of a device 100, there is some possibility that a desired treatment chemical may undesirably interact with the electrolyte, electrode, or a product of the galvanic reaction, thereby compromising a treatment.

FIG. 2 is a schematic block diagram illustrating an alternative embodiment of a disposable device 200 in accordance with the prior art. As a way to increase the voltage between the cationic chamber 104 and anionic chamber 106, a plurality of galvanic cells may be arranged in electrical series on an adhesive strip 102. Two such cells are illustrated in the depicted embodiment. A first electrode 108 in the cationic chamber 104 is connected in series to electrode 202 in cell 204. Electrode 206, also housed in cell 204, is then connected in series to electrode 110 in anionic chamber 106. Such a two cell arrangement can effectively double the voltage generated by the device, and can therefore reduce a length of treatment time required. Additional cells may be added in series, however, the adhesive strip 102 rapidly becomes crowded, thereby limiting the practical range in electromotive force for a device 200.

FIG. 3 is a schematic block diagram illustrating one embodiment of an exploded cross-section view of a disposable device 100. As illustrated, the cationic chamber 104 and anionic chamber 106 typically are open toward the patient. Some sort of substrate 302 typically is provided as a receptor to hold the treatment chemicals (beneficial agent) or electrolyte in a chamber prior to installation of adhesive strip 102 onto a patient. Substrates 302 typically are made from gauze, cellulose, cotton, or other hydrophilic material. It is common practice to saturate the substrates 302 just prior to attaching an adhesive strip 102 to a patient for a treatment session. Substrates 302 may be loaded with treatment substances using a syringe or any other convenient transfer implement.

FIG. 4 is a schematic block diagram illustrating one embodiment of a device 400 driven by a reusable auxiliary power module in accordance with the prior art. A power module 402 typically houses sophisticated electronics, and is relatively expensive (power modules are generally not regarded as single use, disposable items). Power module 402 may provide a substantial voltage to cause ion migration through a body. Applied voltages may reach perhaps 90 Volts, although perhaps for only a very short period of time to initiate ion transfer. Depending upon the skin contact area for ion transfer from a treatment chamber and the composition of the beneficial agent, a patient may perceive a burning sensation under an applied voltage of only 30 volts. Power modules may be attached directly to an adhesive strip 102, as illustrated, but are more commonly connected in-circuit between the cationic chamber 104 and anionic chamber 106 using wires, or extension leads 404, to permit some degree of motion for a patient undergoing a treatment.

The electronics portion of a power module 402 may be constructed to generate a range of voltages, hold a voltage substantially constant for a period of time, or cause a programmable range in voltage over a period of time. Similar modulation may be made by a power module 402 to a current flowing in the circuit. However, power modules 402 represent an expense and may cause inconvenience in that operators may require special expertise to properly configure the module for a particular treatment.

A patient would benefit from a simple, disposable, iontophoretic device capable of higher voltage and more sustained current transmission than currently available disposable devices, but being less costly than devices requiring an electronic or power module. A disposable iontophoretic device having a treatment time operably controlled by the working life of a disposable power source would be an additional advance.

### BRIEF SUMMARY OF THE INVENTION

The invention provides a method for delivering a treatment to a body by way of an iontophoretic transport procedure and device. A device constructed according to principles of the instant invention provides a low cost, disposable, single use, fast and accurate, iontophoretic fluid delivery device for external or implantable use. A body may be construed specifically as a mammalian (e.g. human or animal) body, or alternatively and generally, as a container of an electrolyte. A treatment to be applied to a body by the instant device and method may be either cationic-based, or anionic-based.

An iontophoretic fluid delivery device within contemplation typically includes a cationic chamber, an anionic chamber, and an electromotive force to promote ion exchange between a body and one or both of the chambers. The cationic and anionic chambers define separate volumes in which are held cationic and anionic substances, respectively. A wall of each chamber provides a passageway, or opening, through which ions may migrate. The passageways are generally oriented and arranged on a surface of a container to enable creation of a first conductive path, through a cooperating body, of an electrical circuit between the cationic and anionic chambers.

Treatment materials may be loaded, by syringe or other transfer mechanism, onto a substrate housed within a chamber. Substrates desirably may be configured to reduce polarization of the treatment materials and an attendant drop in reaction rate. One such configuration includes an electrically conductive substrate affixed to a wall of one of the chambers. A workable such substrate may have a surface area, for electron transfer, sized substantially in correspondence with an opening of an ion transfer passageway. An alternate substrate may be formed as electrically conductive gauze. The conductive gauze may be dispersed substantially throughout the volume of the chamber. A hydrogel substance operable as an electrolyte can be disposed, substantially as a pre-loaded item, in one or both of the cationic or anionic chambers. Such a pre-loaded hydrogel can reduce preparation time of a treatment by requiring only the treatment to be introduced, and only to a single chamber of the container.

Devices operable primarily as anionic treatment devices may be made to have a color, texture, shape, or size to differentiate them from a cationic treatment device. Furthermore, individual chambers housed by a container may be made to have different sizes or shapes to facilitate identification and loading of treatment materials into the correct chamber.

One exemplary container can be embodied as an adhesive strip or patch. Alternatively, the container may be a cartridge, carton, or tube for insertion into a body. Devices adapted for insertion into a body, or adapted for storage in preloaded form, may include semipermeable membranes disposed as passageway coverings to contain treatment substances within separate chambers prior to use of a container during a therapeutic treatment.

In one embodiment, the iontophoretic device may use one or more electromotive cells, as required, e.g. to control a length of time for, or rate of, delivery of a quantity of a treatment ion to a body. Such cells may be located partially or completely inside either one or both chambers, or attached to the container in some convenient location.

A cell located partially, or totally, within a chamber generally includes a fluid resistant barrier to isolate an electrolytic path between the cell's positive and negative poles. In such case, a portion of either a positive or a negative pole may be exposed for electron transfer directly to an electrolyte. The cell housing may optionally be formed from, or coated with, a noble or inert metal to avoid its undergoing an undesirable chemical reaction with treatment chemicals. Alternatively, an inert metal may be placed, as an electron interface for the electrochemical reaction, in-circuit between an exterior cell and interior treatment chemicals or fluids. Of course, other conductive metals or alternative conducting materials may be employed in situations where a reaction between the conductive material and treatment fluids would not be detrimental.

One embodiment of the present invention includes a first electromotive cell disposed interior to the cationic chamber. The first cell has an electrolyte barrier exposing only a portion of its negative pole. A second cell, in electrical series with the first cell, may be included interior to the anionic chamber. The second cell also has an electrolyte barrier, but exposing a portion of its positive pole. A conductive path between the two cells is generally sealed to resist transmission of electrolyte from or between the chambers. The invention may alternatively include a single electromotive cell, located in either of the chambers, as desired and practical. In another arrangement, the single electromotive cell may be affixed to container structure separate from both chambers. An embodiment may have electromotive cells located in each chamber, and with one or more additional cells located exterior the chambers and attached to structure of the container. An arrangement of subcells adjacently stacked in electrical series may be regarded as single electromotive cell for purpose of packaging in a chamber, or on a container.

One method of using the present invention, for iontophoretic treatment of a patient, includes the steps of providing an electronic circuit coupling a plurality of electrodes, charging a chargeable electromotive cell to a selected potential in response to a selected quantity of beneficial agent to be delivered, the chargeable electromotive cell being electronically coupled with the electronic circuit, applying the selected quantity of beneficial agent to at least one electrode, placing the at least one electrode in contact with skin, and delivering the selected quantity of beneficial agent. It will be appreciated by those of skill in the art that the electromotive cell can be charged to a selected charge in coulombs as well. In one embodiment, the electrochemical cell is charged to a selected potential corresponding to a number of volts. In another embodiment, the electrochemical cell is charged to a selected charge corresponding to a number of coulombs. It will be appreciated by those of skill in the art that reference to charging the cell to a selected potential or charge may include charging the cell to both a selected potential and a selected charge. It will further be appreciated that charging the electrochemical cell to a selected potential and/or charge, includes charging the electrochemical cell to a selected capacitance and vice versa.

The method may also include attaching a charging circuit to the chargeable electromotive cell, the charging circuit comprising an external electromotive power source, and preparing the skin using a skin preparation device in order to enhance the delivery of the beneficial agent. In one embodiment, preparing the skin comprises puncturing the skin using a micro-needle. In another embodiment, puncturing the skin comprises using a laser. Preparing the skin may also be accomplished by heating the skin or device electrically, chemically, or in other ways.

Other advantages and aspects of the present invention will become apparent upon reading the following description of the drawings and detailed description of the invention. These and other features and advantages of the present invention will become more fully apparent from the following figures, description, and appended claims, or may be learned by the practice of the invention as set forth hereinafter.

### BRIEF DESCRIPTION OF THE SEVERAL VIEWS OF THE DRAWINGS

In order that the manner in which the above-recited and other features and advantages of the invention are obtained will be readily understood, a more particular description of the invention briefly described above will be rendered by reference to specific embodiments thereof that are illustrated in the appended drawings. Understanding that these drawings depict only typical embodiments of the invention and are not therefore to be considered to be limiting of its scope, the invention will be described and explained with additional specificity and detail through the use of the accompanying drawings in which:

FIG. 1 is a top view of a first prior art iontophoretic device;

FIG. 2 is a top view of a second prior art iontophoretic device

FIG. 3 is an exploded side view, in section, of the prior art device depicted in FIG. 1;

FIG. 4 is a top view of a third prior art iontophoretic device;

FIG. 5 illustrates current rate characteristics of certain iontophoretic devices;

FIG. 6 is a top view of an iontophoretic device according to the instant invention;

FIG. 7 is an exploded side view of the device illustrated in FIG. 6;

FIG. 8 is an electric schematic of an iontophoretic circuit;

FIG. 9 is a top view of an alternative embodiment of the invention;

FIG. 10 is a plan view in section of an implantable embodiment of the invention;

FIG. 11 is an electric schematic of an iontophoretic circuit;

FIG. 12 is a plot illustrating cumulative delivery from a device constructed according to the invention compared to a prior art device;

FIG. 13 is a plot of voltage between cationic and anionic chambers during the test illustrated in FIG. 12;

FIG. 14 is an exploded view in perspective of another embodiment of the invention;

FIG. 15 is a schematic view diagram illustrating an alternative embodiment of a device for the transport of treatments into a body in accordance with the present invention;

FIG. 16 is a schematic view diagram illustrating another embodiment of the device for the transport of treatments into a body in accordance with the present invention;

FIG. 17 is a schematic diagram illustrating one embodiment of a skin preparation device in accordance with the present invention; and

FIG. 18 is a schematic flow chart diagram illustrating one embodiment of a method for delivering a beneficial agent into a body in accordance with the present invention.

### DETAILED DESCRIPTION OF THE INVENTION

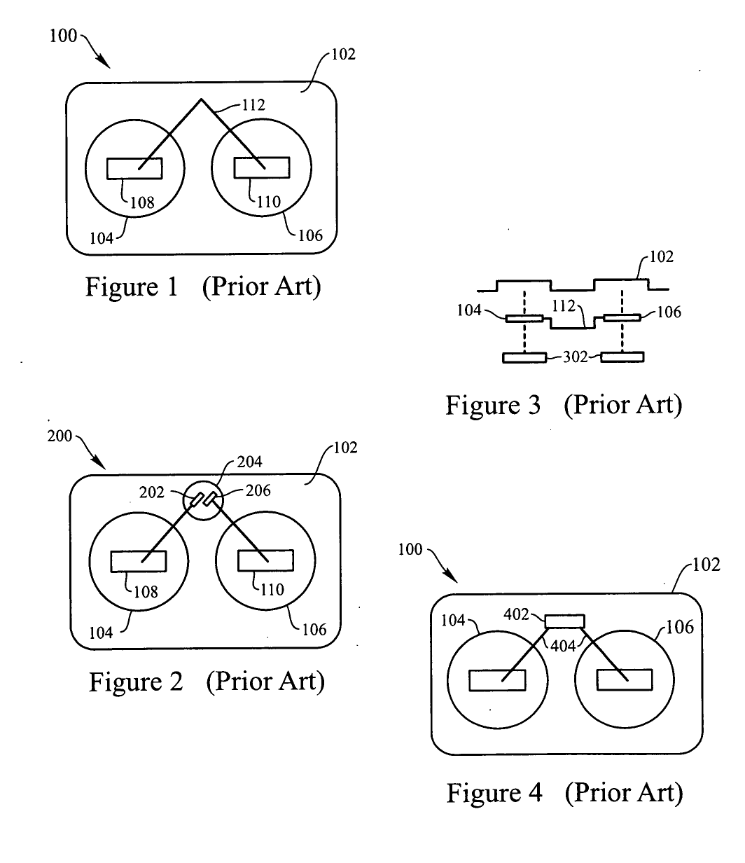

The presented embodiments of the present invention will be best understood by reference to the drawings, wherein like parts are designated by like numerals throughout. It will be readily understood that the components of the present invention, as generally described and illustrated in the figures herein, could be arranged and designed in a wide variety of different configurations. Thus, the following more detailed description of the embodiments of the iontophoretic device of the present invention, as represented in FIGS. 1 through 18, is not intended to limit the scope of the invention, as claimed, but is merely representative of presently preferred embodiments of the invention.

Reference will now be made to the drawings in which the various elements of the invention will be given numerical designations and in which the invention will be discussed so as to enable one skilled in the art to make and use the invention. It is to be understood that the following description is only exemplary of the principles of the present invention, and should not be viewed as narrowing the claims which follow.

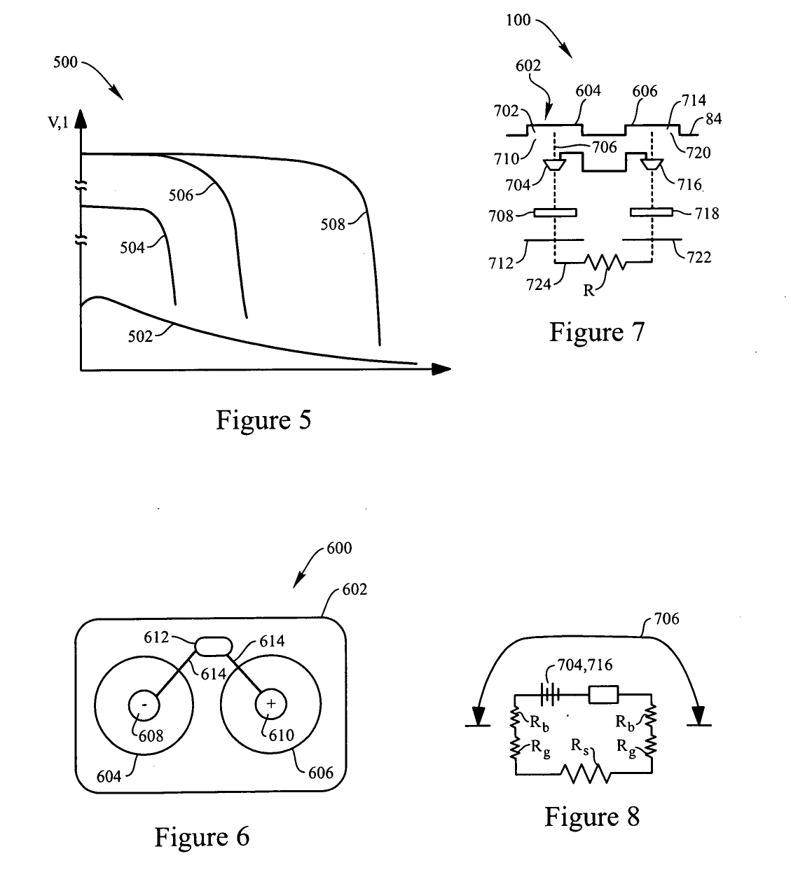

A plot 500 of current discharge or voltage verses time for certain devices is presented in FIG. 5, with the horizontal axis representing passage of time, and the vertical axis showing either a current or available voltage. Trace line 70 is representative of a current profile obtainable in a commercially available and disposable galvanic cell device, such as device 100. Trace line 502 shows a reduced current flow over time due to polarization of electrolyte in the areas surrounding the electrodes, and a corresponding reduced rate of chemical reaction. Trace lines 504-508 are achievable in mini batteries, with the current, or voltage, fall-off occurring when one, or both, reactant is substantially spent in the chemical reaction.

Traces 504-508 illustrate desired current profiles of electromotive cells, such as mini batteries, characterizable as having a substantially “square-wave” over their working life, assuming a sustainable (sufficiently slow) current flow. The working life time of such a mini battery may be controlled to have a desired length by providing only a measured amount of one or more reactant chemicals. The operational life of a mini battery may be set to last 20 seconds, 20 minutes, or multiple hours, simply by controlling the quantity of reactive components in the battery. For example, a battery with the characteristics indicated by trace line 508 may be assembled having about twice as much reactant compared to a battery with the characteristics indicated by trace line 506. A treatment interval may therefore conveniently be determined by the life of a battery. Of course, a treatment time may simply be established by operation by a patient, or by a health care practitioner, of a switch to start and stop a flow of current through the device. Total treatment dose may alternatively also be limited by loading a device with a controlled amount of the ion medicament or beneficial agent.

As indicated by traces 504-508 in FIG. 5, a mini battery also may be constructed to produce a higher voltage than a typical disposable galvanic cell contained in a device 100. A desired voltage may be created by combining oxidizing and reducing agents having sufficient galvanic activity. A battery having the characteristics indicated by trace line 504 would have constituent components with lower combined reactivity than a battery having the characteristics indicated by trace lines 506 and 508. Batteries may also be arranged in electrical series to boost a voltage supplied by a composite cell, effectively forming a more powerful battery. Such a higher voltage may beneficially establish a flow of ions, and cause the ions to migrate at an increased rate to reduce a treatment time requirement. A treatment interval may also be determined, in part, by the voltage of a battery, or effective battery.

Mini batteries may be manufactured having rugged housings to withstand incidental, or even significant, abuse without incurring sufficient damage to suffer a leak of their contents. For purpose of this disclosure, a battery housing is understood to be rugged if the housing is capable of transferring tissue damaging loads to a patient while avoiding a content leaking rupture. A mini battery having a paper housing, for example, would be susceptible to developing a leak which could harm a patient. Such a paper battery is regarded as not being rugged for purpose of this disclosure.

A familiar example for a rugged mini battery type is a button-type battery, which is typically housed in a metal canister resembling a button. Such batteries are commonly employed as power sources for wrist watches. A patient wearing an iontophoretic device incorporating such type of rugged battery would be seriously injured before such a metal button battery would leak due to an object contacting the battery. The rugged housing permits safe use of more reactive materials, such as Lithium, Sodium Hydroxide, and Potassium Hydroxide, with correspondingly higher voltage battery outputs than galvanic reactions using low-concentration electrolyte matched to a human body. Mini batteries are low cost devices, and are available having voltages between about one (1) Volt and about fifteen (15) Volts. The increased voltage provided by a mini battery permits a reduced treatment time in a disposable, single use, iontophoretic device. Rugged mini batteries may also be made in a thin and/or flexible form to reduce bulk of a treatment device. A desirable mini battery for use in the instant invention may be constructed to operate with various metal-anode based electrochemical reactions. Such an anode metal may include Lithium, Zinc, Magnesium, and Aluminum.

Certain embodiments of the present invention differ from the prior art by providing an electromotive force, to drive ion migration, in a self-contained disposable package. A self-contained package may be regarded as providing an electromotive source having a positive pole and a negative pole defined within a single housing. Chemically reactive materials to create a voltage between the positive and negative poles are included inside that housing during manufacture of the electromotive source. The housing is sealed to enclose all of the reactive elements required for electron production. No additional materials, such as electrolyte, must be added subsequent to manufacture of the electromotive source before the source can be used in an electric circuit. Such package structure differentiates over structure of an electromotive source formed by galvanic coupling between a plurality of chambers, such as found in commercially available and disposable iontophoretic devices. A suitable self-contained package to provide such electromotive force can be embodied as a mini battery, including button-cell type mini batteries. Such a mini battery may be the sole electromotive source, or may augment a conventional distributed galvanic reaction arrangement, of a disposable iontophoretic device.

One embodiment of the present invention is illustrated, generally at 600, in FIG. 6. A container 602 spaces apart a cationic chamber 604 and an anionic chamber 606. The chambers are spaced apart to enable creation, with a cooperating body, of an ion conducting path of an electric circuit. The ion conducting path portion of the electric circuit transports the treatment ions into the body.

The container 602 may be sized in correspondence with an area of a patient to be treated. For example, local cosmetic treatment of dark areas under a patient's eyes requires a container sized to attach to a small area. General treatment of a human body with drugs, such as lidocaine, may better be accomplished using the larger surface area available on a patient's shoulder, arm, or area of a torso. Containers 602 may advantageously be formed from a flat and flexible adhesive strip to conform and adhere to a body surface. Containers may also be made in the form of a cartridge, capsule, or tube, for insertion into a body volume.

With continued reference to FIG. 6, a first electromotive cell 608 is located in the cationic chamber 604 of device 600. A second electromotive cell 610 is located in the anionic chamber 606. Cells 608 and 610 may be partially or completely inside the respective chambers. It is further within contemplation for a device according to the present invention to have a single mini battery, which may be located in either of chambers 604 or 606, or simply attached to a container 602. Optional circuit elements 612, nonexclusively including one or more of: an oscillator, a switch, a resistor, a capacitor, an inductor, a transducer, an LED, and the like, may be present in certain embodiments. If present, such components 612 typically are located in an electron conducting path 614 between the cationic chamber 604 and the anionic chamber 606.

A fluid barrier is created on each electromotive cell in illustrated embodiment 600 to prevent a circuit being formed, by electrolyte in a chamber, and carrying current between the individual cell's positive and negative poles. Such a current would detrimentally drain the cell and impede operation of a treatment device 600. One way to create a workable fluid barrier on a pair of mini batteries involves placing the batteries in a die. One battery is placed with its negative pole upwards, and the other battery is placed in the die having its positive pole upwards. The spacing between the batteries in the die should be sufficient to permit location of the batteries as desired in a container 602. A conductor 614 may be attached between both of the upward facing poles, or both of the downward facing poles, by spot welding, or using a conductive adhesive. A preferably inert fluid sealing material, such as an epoxy, plastic, rubber, urethane, or a silicone based product, is then applied to portions of the conductor and mini batteries to form the electrolyte barrier. The barrier forming material may be painted on, sprayed on, or injected into the die. A portion of one pole of each battery is left uncovered by the electrolyte barrier so that one positive pole and one negative pole are exposed for connection in an electric circuit.

Additional details of construction of a representative device 600 are illustrated in FIG. 7. Structure of container 602 spaces apart cationic chamber 604 and anionic chamber 606. Cationic chamber 604 defines a volume 702 in which may be received a mini battery 704, a portion of conductor 706, and a substrate 708. A passageway 710, formed through a wall of chamber 604, provides a path for ion migration toward or away from the chamber 604. In certain embodiments, an optional semi-porous membrane covering 712 may be included to provide a retainer for treatment substances in the chamber 604. Such a covering 712 will be sufficiently permeable to permit ion migration, but desirably will resist fluid flow from the chamber. A chamber covering 712 may be used to enable preloading of a medicant or treatment fluid into a stored device, or in the case where a device is inserted, or placed into, a body.

Still with reference to FIG. 7, anionic chamber 606 defines a volume 714 in which may be received a mini battery 716, a portion of conductor 706, and a substrate 718. A passageway 720, formed through a wall of chamber 606, provides a path for ion migration toward or away from the chamber 606. In certain embodiments, an optional semi-porous membrane covering 722 may be included to provide a retainer for treatment fluids, such as drugs, in the chamber 606, while permitting ion migration through the passageway 720. A device 602 may be attached to a surface of a cooperating body to complete an electric circuit through the body, represented by conductor 724 and resistor R. For purpose of this disclosure, a cooperating body is intended to encompass any structure capable of completing an electric circuit by forming a physical contact spanning between the passageways 710 and 720 to form an ion transporting leg of the circuit. Serviceable bodies include human and animal bodies and other structures which may be considered, in a general sense, to act as containers of electrolyte.

The cationic chamber 604 and anionic chamber 606 typically are formed to define relatively wide and shallow volumes. Passageways 710 and 720 desirably are large to provide a correspondingly large contact area over which ions may migrate into a body. The chamber volumes are generally shallow to minimize a distance, in a depth direction, ions must travel before entering a body. However, polarization of the electrolyte near conducting terminals commonly occurs, and is one source of current reduction depicted by trace 502 in FIG. 5. One way to decrease the polarization effect is to form substrates 708 and 718 to include conductive elements arranged to better distribute electrons though the chamber volume. In any case, a distributed current transmission is desirable in both types of iontophoretic devices. A desirable distribution may limit an effective sustained current density over a treatment area to less than about 0.5 mA/cm·sup·2 to reduce the chance of a patient experiencing skin irritation during a treatment.

Substrates 708 and 718 may include conductive material affixed to a wall area of one or both chambers. Such conductive material may be painted, sprayed, or otherwise affixed to a portion of, or on the entire inside surface of, a chamber. Desirably, such conductive material will encompass an area opposite, and sized in agreement with, a passage opening 710 or 720. Alternatively, a substrate 708 or 718 may be formed from a conductive material and distributed through a volume 702 or 714. A workable distributed substrate may be formed by impregnating a conventional substrate, such as a gauze, with a conductive substance, such as a metal powder. A substrate also may be made from a metal or metal/polymer composite.

FIG. 8 illustrates an electric schematic of an iontophoretic reaction process. Resistances to current flow in such a circuit include Rb, for resistance through the battery, R_{g}, for resistance through the gauze or from a terminal to an electrolyte, and Rₛ, representing skin resistance of a body. Polarization of the constituent chemicals in a conventional disposable galvanically driven iontophoretic reaction tends to increase a value for R_{g}, and decrease a transmitted current. Distributing a surface for electron transfer through a volume of a chamber tends to counter onset of such polarization. The benefit to a patient undergoing a treatment with improved electron distribution is an increased and consistent ion delivery rate, permitting a reduced treatment time interval.

It is desirable for conductors 706, and exposed portions of electromotive cells to not detrimentally react with treatment chemicals in a chamber 604 or 606. A detrimental reaction would decrease efficacy of the treatment, or may form a caustic or noxious substance which might irritate a patient's skin. One way to resist such undesired chemical interaction is to provide a mini battery or electromotive cell with an inert housing, or coating. An exposed electron exchange surface portion may be made from, or coated with, a chemically inert conductor or noble conductive material. For purpose of this disclosure, a noble conductor can be defined as a material serviceable to conduct electrons, but otherwise generally nonparticipatory in a chemical reaction with substances in which it is immersed or contacting. Examples of such noble conductive materials nonexclusively include molybdenum, gold, silver, carbon, titanium, and tantalum. As an additional precaution, a battery may be located external to a chamber, and electrically connected to a noble conductor located inside a chamber for electron exchange.

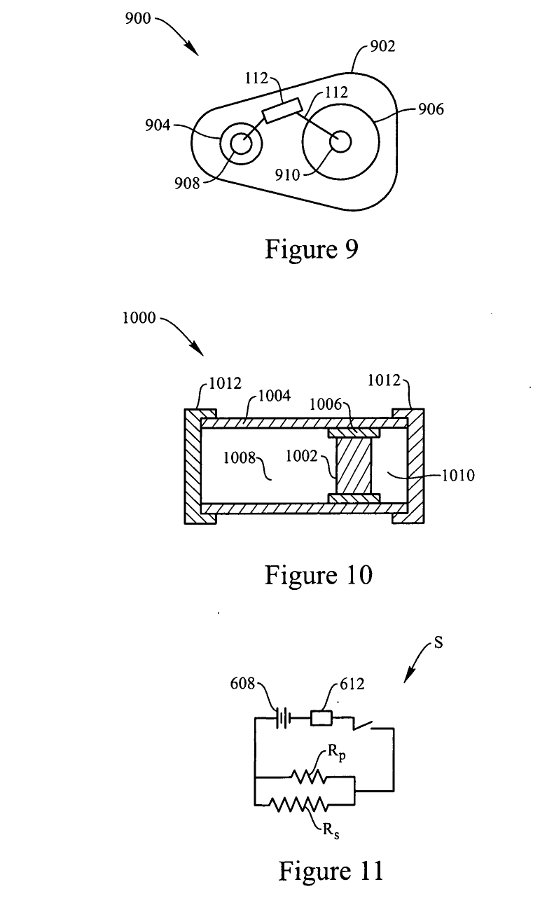

Iontophoretic devices according to the instant invention, such as indicated generally at 900 in FIG. 9, may be constructed having a different size or shape between cationic and anionic chambers. The shape, color, texture or some other discernable characteristic of container 902 may also be used as an indicator of the device's use for cationic or anionic treatments. For example, a red container 902 may signify that the device 900 is for use to dispense anionic-based treatments. A yellow container 902 may signify that the device 900 is adapted to dispense cationic-based treatments. For convenience, a chamber 904 may be preloaded for storage with a hydrogel capable of acting as an electrolyte. A treatment drug then need only be loaded into chamber 906 prior to placing the container 902 onto a body. The shape and/or size difference between chambers 904 and 906 can assist in loading the treatment into the correct chamber to establish treatment ion migration directed toward the body. Of course, an exposed, or electrolytically connectable, pole of each of batteries 908 and 910 will have an appropriate electrical sign, depending on the construction and desired purpose of the device 900 as either a cationic or anionic beneficial agent dispensing device.

One embodiment of an implantable iontophoretic device according to the instant invention is illustrated generally at 1000 in FIG. 10. In one use, device 1000 may be surgically implanted into a body to provide a long-term pain treatment. A device 1000 has an electromotive source 1002 housed in a container 1004. Illustrated container 1004 is constructed as a cylinder. A barrier 1006, adapted to prevent an electrolytic circuit between positive and negative poles of source 1002, is included when the electromotive source 1002 includes one or more mini batteries. Barrier 1006 may be adapted sealingly to slide like a piston inside container 1004 to accommodate a change in chamber volume due to transfer of ions from a chamber containing a beneficial treatment agent. Alternatively, a container can be constructed directly to expand or contract and thereby accommodate changes in chamber volume. Chamber 1008 can be a cationic chamber when chamber 1010 is an anionic chamber. Of course, reversing the polarity arrangement of the electromotive source 1002 will reverse each chamber's role. Some sort of semipermeable cap 1012 is provided to cover openings from the respective chambers. Suitable caps 1012 permit migration of ions in and out of the chambers, but otherwise resist unintended leaking of chamber contents.

Commercially available mini batteries typically provide a higher capacity, or contain more stored energy, than required to dispense a desired ion dose of a beneficial agent. A device according to the present invention may be adapted accurately to dispense a controlled dose of beneficial treatment by incorporating a suitable circuit arrangement in the electron carrying portion of the device's electric circuit. An electric circuit may be arranged to direct virtually any portion of an electromotive source's available stored energy, from zero to 100 percent, to ion transport.

One way to apportion a source's stored energy is illustrated in FIG. 11. A shunt resister, Rₚ, can be connected in-circuit to form an electron conducting path in parallel to the ion conducting path through a body. A representative switch S can conveniently be closed by loading a chamber with electrolyte and application of a device to a body to complete the circuit. As is well known in electric circuit design, the current flow through the shunt resister Rₚ and the body resistance Rₛ will be determined by the relative magnitude of the resistance in each path. Decreasing the value for Rₚ increases the current flow through the parallel path, and decreases the current flow through the ion conducting path, resulting in a lower dispensed beneficial agent ion dose. A device may therefore be constructed to deliver a dose of ion-based treatment corresponding to any portion of a battery's capacity.

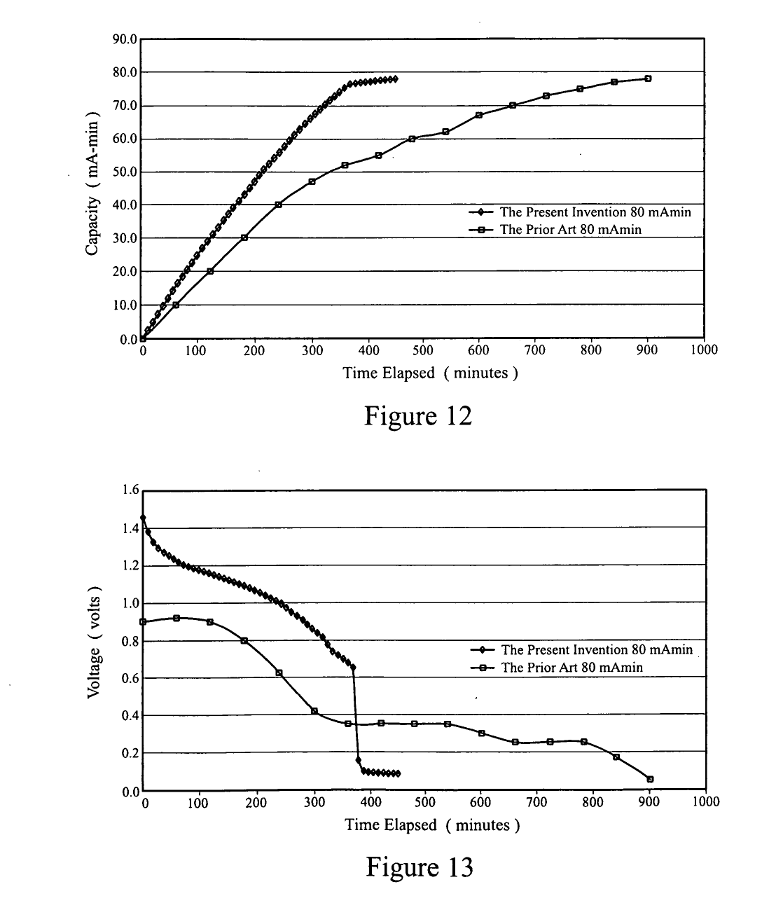

The dispensed ion dose will directly correspond to the current flow through the ion conducting path. FIGS. 12 and 13 illustrate the performance of a device constructed according to the instant invention compared to a comparable device constructed according to the teachings of the prior art. One prior art of device was made by forming electrodes from Zn and AgCl. The present invention was embodied with a single 1.5 volt button battery rated at 900 mAmp-min (milliamp-minutes). Skin resistance Rₛ was modeled with a 5 k-ohm resister. The shunt resister Rₚ was 500 ohms. Useful shunt resistances may range from 1 ohm to about 10,000 ohms, or more.

With reference to FIG. 12, it may be seen that the invention delivered a current corresponding to an equivalent dose of beneficial agent totaling about 78 mAmp-min in about 400 minutes. The prior art device required over 850 minutes, or more than twice as long, to accomplish the same dose.

FIG. 13 illustrates the voltage measured between the cationic and anionic chambers during the test illustrated in FIG. 12. It may be noted, with reference to FIG. 13, that the trace of voltage over time for the invention is not a perfectly “square” square-wave shape. That is, the voltage drops over time, instead of remaining relatively constant for about the first 375 minutes. The current discharge through both the shunt and skin paths exceeds the battery's steady state discharge rate at which battery voltage may remain relatively constant. However, the voltage does exhibit a sharp drop as the battery approaches full discharge. The battery inherently expends its energy more rapidly and uniformly than the electrolytic cell, and does so up to substantially complete exhaustion. Such a characteristic is desirable as one way accurately to control a treatment dose. The device according to the invention provides a disposable iontophoretic apparatus which is faster in delivering a treatment dose and also more precise in termination of the treatment interval.

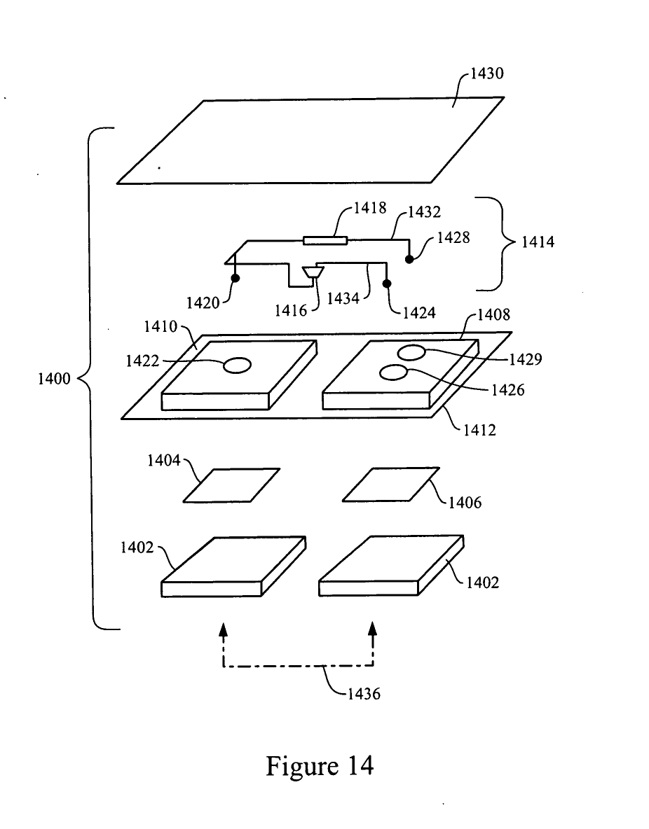

One way to manufacture a device to include a shunt resistance in a parallel path between the cationic and anionic chambers is illustrated generally at 1400 in FIG. 14. Substrates 1402 and electrodes 1404, 1406 are housed in chambers 1408 and 1410. Chambers 1408 and 1410 are formed in container 1412, which may beneficially have an adhesive coating on one surface. Circuit elements, generally indicated at 1414, are placed on top of container 1412. Circuit elements can include a battery 1416, and a component assembly 1418. The battery 1416 and component assembly 1418 are electrically connected at junction 1420, through aperture 1422, to electrode 1404. Battery 1416 is connected at junction 1424 to electrode 1406 through aperture 1426 in chamber 1408. Component assembly 1418 has terminal 1428 disposed through port 1429 in chamber 1408, but away from contact with electrode 1406. An electrical circuit is formed between terminals 1424 and 1428 only after introduction of an electrolyte to chamber 1408. The electrolyte effectively acts as a switch in-circuit with the battery 1416 and component assembly 1418. A protective top cover 1430 desirably is placed over the components 1414 to provide a pleasing appearance.

Still with reference to FIG. 14, it is within contemplation for component assembly 1418 to include one or more of: an oscillator, a switch, a resistor, a capacitor, an inductor, a transducer, a Light Emitting Diode (LED), circuitry and elements formed on a silicone chip, and the like. The location for placement of alternative circuit elements may be manipulated to optimize the device for efficacy, manufacturability, and patient comfort.

In an embodiment having an LED, an appropriate aperture, or window for light transmission, is provided in the covering 1430, if present. The covering 1430 can also be transparent. An LED may be placed in the conductive path 1432 between junction 1420 and junction 1428 to provide a visual indicator showing status of the treatment. In the arrangement illustrated in FIG. 14, the battery is connected by circuit path 1434 to parallel electron conducting path 1432 and ion conducting path 1436. If current is flowing in path 1432, it should also be flowing in path 1436, and delivering a dose of ions to a patient. Therefore, when an LED begins to produce a visible output, a patient can be confident that the treatment is proceeding. When the LED no longer produces a visible output, the patient can be confident that the treatment is concluded. An LED can be sized to draw electrical energy from a battery 1416 at a rate to deplete a particular battery 1416 in a desired time interval, and thereby operate to control a treatment interval.

An oscillator element disposed in-circuit in the conductive path 1432 can operate to control a current flow between high and low values. A pulse-delivery of certain treatment agents may enhance such delivery over a steady-state type of delivery. Additional benefits may accrue to a patient from a massaging effect of the pulse. A manual or automatic switch placed in the second path may be used to start and stop treatments at controlled time intervals.

An electronic component capable of dissipating electric energy in the form of heat (e.g. a resistor) may advantageously be placed in a position operable to heat the contents of a chamber, such as a treatment fluid. Warming the treatment fluid or agent can increase a rate of reaction or solubility of a treatment substance, improving efficacy of the device. A shunt resistance in a parallel circuit to the iontophoretic path 1436 of the circuit may control delivery of a beneficial agent in an amount over a time interval corresponding to any portion of a battery 1416 capacity, typically between about 1 mAmp-min to 500 mAmp-min, or more. An LED is one alternate electric component that can perform the same dosing function, and can also operate to dissipate electrical energy as heat to warm a chamber's contents.

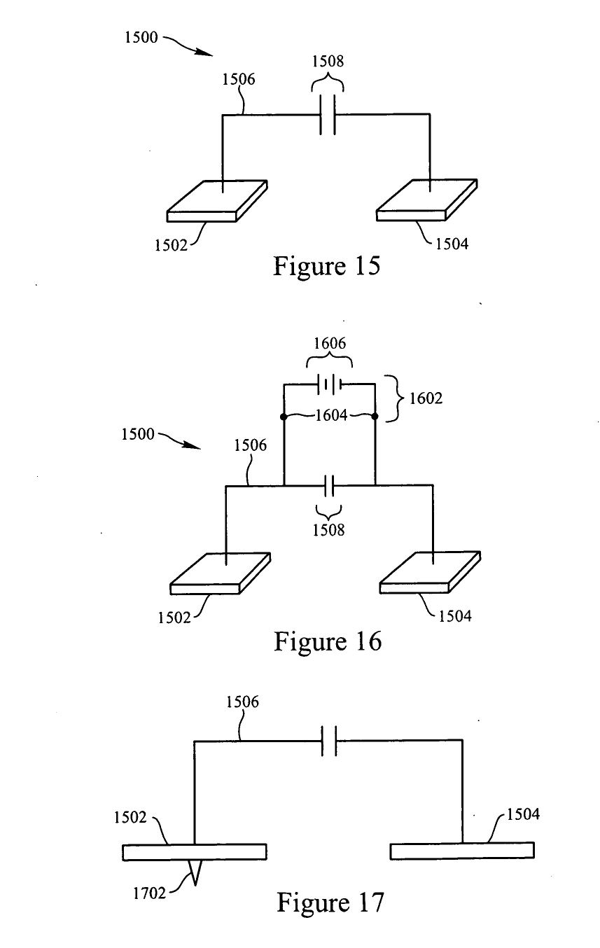

FIG. 15 is a schematic view diagram illustrating an alternative embodiment of a device 1500 for the transport of treatments into a body in accordance with the present invention. In one embodiment the device comprises electrodes 1502, 1504 electronically coupled via a conductor 1506. In a further embodiment, an electromotive cell is disposed between the electrodes 1502, 1504. As described above, the electromotive cell may comprise a battery. Alternatively, the electromotive cell may comprise a capacitor 1508 as depicted in FIG. 15. The capacitor may be formed of a dielectric capacitor or electrochemical (or electrolytic) capacitor.

The capacitor 1508 may be selected having a charge and voltage capacity selected according to the quantity of treatment agent or fluid to be delivered into the body. The capacitor 1508, in one embodiment, may be able to deliver about 5 to about 500 mAmp-min of charge to the skin, depending on the amount of beneficial agent to be delivered. Additionally, the potential of the capacitor 1508 is in the range of between about 1 V and 60 V. In one embodiment, the potential of the capacitor 1508 is in the range of between about 20 V and about 40 V. The ranges given above are selected according to a current required to “drive” the beneficial agent into the body of a patient.

FIG. 16 is a schematic view diagram illustrating another embodiment of the device 1500 for the transport of treatments into a body in accordance with the present invention. In one embodiment, the capacitor 1508 may be charged by a charging circuit 1602 removably coupled to the device 1500 and configured to charge the capacitor 1508 to a desired capacitance and voltage. The charging circuit 1602 may comprise attachable leads 1604 for electronically connecting to the device 1500. The charging circuit 1602 also includes a charging source, which in one embodiment comprises a DC battery 1606.

The battery 1606, in one example comprises a simple 9V battery. Once the battery 1606 is connected with the device 1500, the capacitor 1508 is charged until the potential of the capacitor 1508 is equivalent to the potential of the battery 1606. Alternatively, the charging source may comprise an external power supply such as an AC or DC power supply, different voltage batteries, or a second capacitor.

FIG. 17 is a schematic diagram illustrating one embodiment of a skin preparation device 1702 in accordance with the present invention. The skin preparation device 1702, in one embodiment, comprises a micro-needle. The skin preparation 1702 extends downward from the substrate or electrode in order to puncture the surface of the skin or stratum corneum in order to enhance the delivery of the beneficial agent into the body. The skin preparation device 1702 overcomes the problem of each person having a different skin resistance.

Certain people may have such a high skin resistance that the device 1500 is not able to effectively deliver the beneficial agent into the body. However, once the surface of the skin is broken, the internal resistance of the body is substantially similar across different races, ages, genders, etc. A consistent resistance enables the use of a single capacitance and voltage for a specified quantity of beneficial agent. In a further embodiment, the skin preparation device 1702 may comprise a laser, a drill, or any device capable of puncturing, perforating, or making an opening in the skin. The skin preparation device may also be a heating unit.

The schematic flow chart diagram that follows is generally set forth as a logical flow chart diagram. As such, the depicted order and labeled steps are indicative of one embodiment of the presented method. Other steps and methods may be conceived that are equivalent in function, logic, or effect to one or more steps, or portions thereof, of the illustrated method. Additionally, the format and symbols employed are provided to explain the logical steps of the method and are understood not to limit the scope of the method. Although various arrow types and line types may be employed in the flow chart diagrams, they are understood not to limit the scope of the corresponding method. Indeed, some arrows or other connectors may be used to indicate only the logical flow of the method. For instance, an arrow may indicate a waiting or monitoring period of unspecified duration between enumerated steps of the depicted method. Additionally, the order in which a particular method occurs may or may not strictly adhere to the order of the corresponding steps shown.

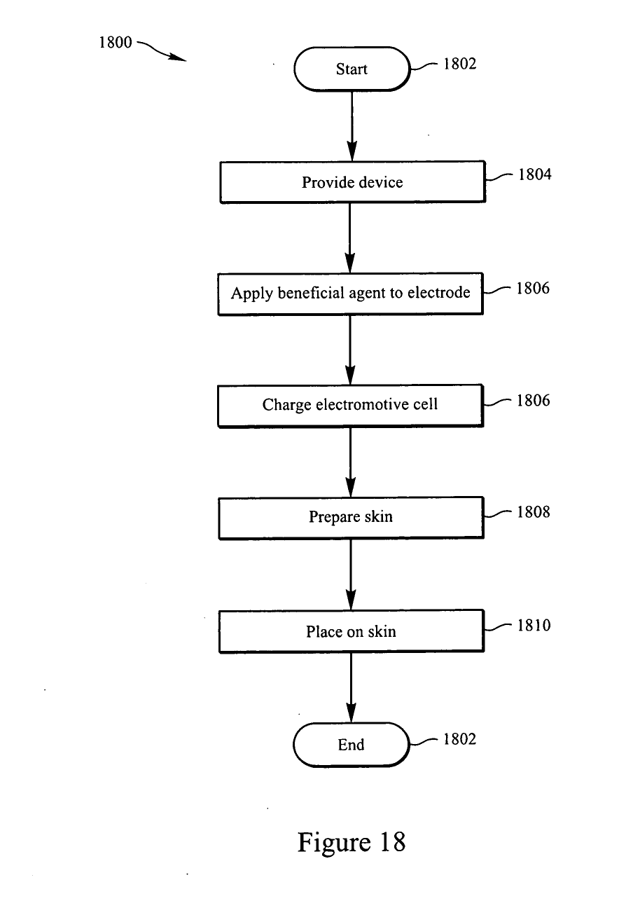

FIG. 18 is a schematic flow chart diagram illustrating one embodiment of a method 1800 for delivering a beneficial agent into a body in accordance with the present invention. In one embodiment, the method 1800 starts 1802 and a device is provided 1804 in accordance with the present invention. For example, a device such as the device 1500 of FIG. 15 is provided having electrodes and an electromotive cell, e.g. a capacitor. The capacitor may comprise a dielectric or electrochemical capacitor. The method continues and the beneficial agent is applied 1806 to the device as described above.

Upon applying 1806 the beneficial agent, a charging circuit 1602 may be connected to the device 1500 in order to charge 1806 the electromotive cell. For example, assume a 9V battery is attached to the device 1500. The charging circuit will raise the potential and/or charge of the capacitor until the potential or charge of the capacitor is equivalent to the charging circuit. The skin preparation device 1702 then prepares 1808 the skin to receive the device 1500. In one embodiment, preparing 1808 the skin comprises puncturing the skin with a micro-needle. Alternatively, preparing the skin may comprise puncturing, perforating, or creating an opening in the skin. In one embodiment, preparing the skin comprises heating the skin. This could be accomplished electronically, chemically or in other ways or combinations of ways. The skin could be heated directly or by heating the device 1500. The device 1500 may then be placed 1810 on the skin. The method 1800 then ends 1812.

While the invention has been described in particular with reference to certain illustrated embodiments, such is not intended to limit the scope of the invention. The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1-28. (canceled)

29. An iontophoresis patch for delivering a beneficial agent through the skin of a patient, said patch comprising:
(a) an adhesive strip having on one side thereof a therapeutic face releasable securable to the skin of the patient;
(b) an absorbent substrate carried on a portion of said therapeutic face of said adhesive strip and capable of retaining therein a predetermined quantity of a liquid containing the beneficial agent, said predetermined quantity of the liquid retained in said absorbent substrate electrically contacting the skin of the patient, when said adhesive strip is releasably secured thereto;
(c) a first electrode carried on said adhesive strip electrically conductive engaging said predetermined quantity of the liquid retained in said absorbent substrate;
(d) a second electrode carried on said adhesive strip in electrical isolation from said first electrode and said absorbent substrate, said second electrode electrically conductive engaging the skin of the patient when said therapeutic face of said adhesive strip is releasably secured thereto; and
(e) a charged capacitor carried on said adhesive strip electrically interconnecting said first electrode and said second electrode, said charged capacitor causing electrical current to flow from said predetermined quantity of the liquid retained in said absorbent substrate through the skin of the patient to said second electrode, when said therapeutic face of said adhesive strip is releasably secured to the skin of the patient.

30. An iontophoresis patch as recited in claim 29, wherein said charged capacitor comprises a dielectric capacitor.

31. An iontophoresis patch as recited in claim 29, wherein said charged capacitor comprises an electrochemical capacitor.

32. An iontophoresis patch as recited in claim 29, wherein said first electrode in and said absorbent substrate function as a cationic chamber relative to said predetermined quantity of the liquid.

33. An iontophoresis patch as recited in claim 29, wherein said first electrode and said absorbent substrate function as a anionic chamber relative to said predetermined quantity of the liquid.

34. An iontophoresis patch as recited in claim 29, further comprising selectively engageable charging contacts on opposite sides of said capacitor, charge for said capacitor being delivered to said capacitor through said charging contacts.

35. An iontophoretic system for delivering a beneficial agent through the skin of a patient, said system comprising:
(a) an iontophoresis patch releasably securable to the skin of the patient by a therapeutic face thereof, said patch comprising:
(i) an absorbent substrate carried on a portion of said therapeutic face of said adhesive strip capable functioning as a reservoir for a predetermined quantity of a liquid containing the beneficial agent, said absorbent electrically contacting the skin of the patient with said predetermined quantity of the liquid, when said iontophoresis patch is releasably secured thereto;
(ii) a first electrode carried on said iontophoresis patch electrically conductive engaging said predetermined quantity of the liquid retained in said absorbent substrate;
(iii) a second electrode carried on said iontophoresis patch in electrical isolation from said first electrode and said absorbent substrate, said second electrode electrically conductive engaging the skin of the patient when said therapeutic face of said iontophoresis patch is releasably secured thereto;
(iv) a capacitor carried on said iontophoresis patch electrically interconnecting said first electrode and said second electrode; and
(v) charging contacts on opposite sides of said capacitor; and
(b) a charging circuit removably electrically engageable across said charging contacts, said charging circuit delivering to said capacitor a predetermined charge correlated to a predetermined dose of the beneficial agent to be delivered iontophoretically into the skin of the patient.

36. An iontophoretic system as recited in claim 35, wherein said charging circuit comprises:
(a) a charging source of said predetermined charge; and
(b) leads electrically coupled to said charging source, said leads being selectively electrically engageable, respectively, to individual of said charging contacts.

37. An iontophoretic system as recited in claim 35, wherein said capacitor comprises a dielectric capacitor.

38. An iontophoretic system as recited in claim 35, wherein said capacitor comprises an electrochemical capacitor.

39. An iontophoretic system as recited in claim 35, wherein said first electrode in and said absorbent substrate function as a cationic chamber relative to said predetermined quantity of the liquid.

40. An iontophoretic system as recited in claim 35, wherein said first electrode and said absorbent substrate function as a anionic chamber relative to said predetermined quantity of the liquid.

41. A method for delivering a beneficial agent through the skin of a patient, said method comprising the steps of:
(a) mounting a first electrode and a second electrode in electrical isolation on an adhesive strip having on one side thereof a therapeutic face releasable securable to the skin of the patient, said second electrode electrically conductive engaging the skin of the patient when said therapeutic face of said adhesive strip is releasably secured thereto;
(b) positioning an absorbent substrate on said therapeutic face of said adhesive strip in electrical communication with said first electrode electrically conductive engaging the skin of the patient when said therapeutic face of said adhesive strip is releasably secured thereto:
(c) electrically coupling said first electrode to said second electrode through a capacitor;
(d) delivering onto said capacitor a predetermined charge correlated to a predetermined dose of the beneficial agent to be delivered iontophoretically into the skin of the patient;
(e) administering into said absorbent substrate a predetermined quantity of a liquid containing the beneficial agent, said absorbent electrically contacting the skin of the patient with said predetermined quantity of the liquid when said therapeutic face of said adhesive strip is releasably secured thereto; and
(f) engaging the skin of the patient with said therapeutic face of said adhesive strip.

42. A method as recited in claim 41, wherein said step of delivering onto said capacitor a predetermined charge comprises the step of removably electrically engaging across said capacitor a charging circuit comprising:
(a) a charging source of said predetermined charge; and
(b) leads electrically couples to said charging source, said electrical leads being selectively electrically engageable, respectively, to opposite sides of said capacitor.
